(19) United States
(12) Patent Application Publication
(10) Pub. No.: US 2010/0255062 A1
(43) Pub. Date: Oct. 7, 2010

(54) COMPOUNDS, POLYMERS AND METHODS FOR TREATING GASTROINTESTINAL DYSFUNCTION

(75) Inventors: Aristotle G. Kalivretenos, Columbia, MD (US); Robert E. Raulli, Baltimore, MD (US); Niaz Sahibzada, Baltimore, MD (US); Richard Gillis, Baltimore, MD (US); Mark S. Niedringhaus, Baltimore, MD (US)

Correspondence Address: FOLEY AND LARDNER LLP;SUITE 500, 3000 K STREET NW, WASHINGTON, DC, 20007, US

(73) Assignee: AMULET PHARMACEUTICALS, INC.

(21) Appl. No.: 12/602,198

(22) PCT Filed: Jun. 2, 2008
(86) PCT No.: PCT/US08/06935

§ 371 Date: Jun. 14, 2010

Related U.S. Application Data
(60) Provisional application No. 60/924,830, filed on Jun. 1, 2007.

(51) Int. Cl. A61K 9/68; A61K 47/48; A61K 9/50; A61P 1/00

(52) U.S. Cl. 424/440; 424/78.17; 424/490

(57) ABSTRACT

The present disclosure describes novel carbon-based diazeniumdiolates agents and compounds or salts or prodrugs thereof that release nitric oxide for the treatment of neuropathic gastrointestinal dysfunction. The neuropathic gastrointestinal dysfunction refers to disorders associated with motility, sensation and neuromuscular function that include but are not limited to conditions such as delayed gastric emptying.

### FIELD OF THE INVENTION

The present invention relates to compositions, polymers and methods for treating gastrointestinal dysfunction. More particularly, the present invention relates to compositions, polymers and methods using nitric oxide to treat neuropathic gastrointestinal dysfunction.

### BACKGROUND OF INVENTION

One type of gastrointestinal dysfunction is neuropathic gastrointestinal dysfunction, which refers to disorders of motility, sensation and neuromuscular function. Neuropathic gastrointestinal dysfunction may occur in, for example, diabetic patients where the signs or symptoms may share underlying molecular causes whether or not delayed gastric emptying is observed (Shah et al., 2004). This disorder can occur in Type I diabetes with or without peripheral neuropathy, and it also can be a complication resulting from Type II diabetes. Gastrointestinal dysfunction can manifest itself in delayed gastric emptying (GE), which is also known as gastroparesis. According to the American Gastroenterological Association Medical Position Statement: Diagnosis and treatment of gastroparesis (Parkman et al., 2004), “gastroparesis is a symptomatic chronic disorder of the stomach characterized by delayed GE in the absence of mechanical obstruction.” Symptoms include early satiety, nausea, bloating, vomiting and abdominal pain or discomfort. Gastroparesis is a common and debilitating condition affecting millions of patients with diabetes. Current treatments for gastroparesis include metoclopramide, erythromycin, cisapride and domperidone. Although all of these agents are employed as promotility agents to improve the number and intensity of gastric contractions, they have not been shown to be uniformly effective in controlled clinical studies. Furthermore, these agents possess unwanted side effects that limit their use. In particular, cisapride is only available through a special program due to its propensity to produce QT prolongation resulting in ventricular arrhythmias. Metoclopramide possesses antiemetic as well as prokinetic effects but its clinical utility is limited by adverse central nervous system effects. Long term use can result in extrapyramidal side effects, tardive dyskinesia, akathisia, drowsiness, depression, impotence and hyperprolactinemia. Erythromycin has been associated with cramping, nausea, diarrhea and vomiting as well as potentially causing ventricular arrhythmias as a result of QT prolongation. The prokinetic effect of erythromycin develops rapid tachyphylaxis, thereby limiting the utility of the drug. Domperidone is a peripheral D₂ antagonist that does not demonstrate central nervous system side effects. However, its effectiveness is equivocal and it has not been approved in the United States.

After receiving nutrients, the stomach will grind, mix and empty its contents into the small bowel where it is absorbed. These functions are coordinated thorough the central, autonomic and enteric nervous system. Nitric oxide (NO) has been shown to regulate several of the essential events that enable normal gastric emptying to occur. These events include relaxation of the fundus to accommodate food, contractions of the antrum for breakdown of gastric contents, relaxation of the pyloric sphincter to allow gastric contents to exit the stomach, and orderly coordination of antropyloroduodenal activities (Parkman et al., 2004; Shah et al., 2004). Clinical data indicate that normal relaxation of the pyloric sphincter is essential for the coordinated antropyloroduodenal muscle activity that underlies normal GE (Horowitz et al., 1994).

Evidence from several investigators indicates that loss of neuronal nitric oxide synthase (nNOS) and therefore loss of NO in select regions of the stomach such as the pylorus, results in delayed GE (Micci et al., 2005, Watkins et al., 2000). Indeed, mice with targeted disruption of the nNOS gene exhibit hypertrophy of the pyloric sphincter, enlargement of the stomach and delayed GE (Watkins et al., 2000). Importantly, this occurs without loss of myenteric neurons in the pylorus or without loss of vasoactive intestinal polypeptide (VIP), the other major gastrointestinal (GI) inhibitory transmitter. Studies of ex vivo organ bath preparations of the pyloric sphincter muscle reveal that electrical field stimulation (EFS) produces NO-mediated relaxation of the sphincter from wild type mice, which is absent in the sphincter from mice with targeted genomic deletion of nNOS (Watkins et al., 2000). These results further demonstrate the role of NO in normal gastric emptying and establish targeted genomic deletion of neuronal nNOS as a genetic model of gastroparesis (Micci et al., 2005). Pharmacological inhibition of nNOS in experimental animals has also been demonstrated to slow GE (Orihata and Sarna, 1994). Clinically, it has been shown that the inability of the pyloric sphincter to relax normally contributes to diabetic gastroparesis (Mearin et al., 1986).

Given these data, restoring nitrergic neurotransmission to the stomach should significantly improve gastric function in animals with experimentally induced diabetic gastroparesis. This has been shown to be the case in experimental animal studies (Micci et al., 2005, Watkins et al., 2000). Preliminary data from diabetic gastroparesis patients also suggest a positive benefit from the drug sildenafil (Bianco et al., 2002) which prevents the breakdown of cGMP, the mediator of NO's effect on gastric smooth muscle. Insulin treatment for one week of animals with experimentally induced diabetic gastroparesis will restore both pyloric NOS levels and GE to normal (Watkins et al., 2000).

Additional evidence for the role of nitric oxide in normal gastric function was obtained through an in vivo recording of pyloric sphincter muscle activity measured using a strain gauge force transducer sutured along the circular muscle of the sphincter of the rat (Ishiguchi et al., 2001). The vago-vagal reflex was activated by placing an intragastric balloon and inflating it in a way to primarily distend the antrum. Distension resulted in a vago-vagal reflex mediated relaxation of the pyloric sphincter. This pyloric relaxation was abolished by treating animals with a pharmacologic inhibitor of NOS. The investigators suggested that gastric distension-induced pyloric relaxation was mediated via a vago-vagal reflex and NO release (Ishiguchi et al., 2001) demonstrating the role of NO in vago-vagal reflex control of the pyloric sphincter.

A deficiency in nitric oxide has been demonstrated to lead to esophageal dysfunction. Nitric Oxide is a relaxatory neurotransmitter in the esophagus and lower esophageal sphincter. Blockade of NO synthesis reduces the latency between swallows, causes contraction in the distal esophagus, increases basal LES pressure, increases peristaltic wave pressure, and decreases the number of transient LES relaxations (Sivarao et al., 2001).

Achalasia is a disorder of the esophagus where food is less able to move toward the stomach due to the insufficient relaxation of the muscle from the esophagus to the stomach after swallowing. This relaxation is needed to allow food to enter the stomach. Patients with achalasia display an absence of nNOS immunoreactivity and enzymatic activity in LES neurons (Mearin et al., 1993). Additionally, nNOS^{-/-} mice display elevated baseline LES pressures and reduced swallow-induced relaxation of the LES. The phosphodiesterase type-5 inhibitor, Sildenafil, has been used to potentiate the NO-cGMP pathway to decrease the swallow-induced contractions in the distal esophagus in patients with esophageal motor disease. Although patients with achalasia did not appear to benefit from treatment, there was symptomatic improvement in some patients with nutcracker esophagus, esophageal spasm and hypertensive LES following chronic administration (Eherer et al., 2002).

Thus, there is a need for novel therapies that facilitate nitric oxide signaling and offer a clear benefit in the treatment of gastrointestinal dysfunction. These new therapies would provide the distinct advantages produced by nitric oxide without suffering from the drawbacks characteristic of conventional therapies.

### SUMMARY OF THE INVENTION

The present invention describes compositions and methods for treating gastrointestinal dysfunction using nitric oxide releasing agents or polymers. Neuropathic gastrointestinal dysfunction refers to disorders of motility, sensation and neuromuscular function. The nitric oxide (NO) releasing polymers are carbon-based or C-based diazeniumdiolates or salts or prodrugs thereof that are specifically designed to release nitric oxide under physiological conditions present in the gastrointestinal tract and thus minimize systemic exposure to nitric oxide. The invention also presents methods for using nitric oxide releasing agents or polymers for treating gastrointestinal disorders that result in but are not limited to delayed gastric emptying such as gastroparesis. In addition, the invention includes the geometric isomers, enantiomers, diastereomers, and pharmaceutically acceptable salts thereof of the described compounds and methods of their use in treating gastrointestinal dysfunction.

### BRIEF DESCRIPTION OF THE DRAWINGS

FIG. 1 shows a comparison of NO release from the diazeniumdiolated cross-linked acetylpolystyrene at physiologic (7.4) and gastric pH (2.1).

### DETAILED DESCRIPTION OF THE INVENTION

The present invention provides novel compounds to deliver nitric oxide to the gastrointestinal tract to treat diseases that are mediated by a reduction or absence of endogenous nitric oxide. Also provided are methods for controlling the rate of nitric oxide delivery and the localization of this active agent to the site(s) of dysfunction.

### Use of Dissolved NO Gas as a Dosage Form to Treat Diabetic Gastroparesis

A dose of nitric oxide to treat gastroparesis can be delivered in multiple ways, including but not limited to delivery as a solution in water or aqueous dosage form, among others. A solution of NO in degassed water can be made that will be relatively stable in a cold, oxygen-free environment for weeks. One skilled in the art will be cognizant of a variety of conventional degassing methods that can be used, and will have knowledge of methods to introduce NO gas into degassed water under an inert, oxygen fee environment. The aqueous NO solution of the appropriate concentration can be packaged in a hermetically sealed container that will remain sealed at 4° C., is easy to open and can deliver the dose directly to the patient by, for example, bringing the container to the lips and drinking directly from the container. One non-limiting exemplary embodiment is not unlike the tear and pour mini-creamer containers found at a coffee shop. One skilled in the art can envision a variety of ways to properly contain the aqueous NO dosage form, including but not limited to sealed ampules, liquid-filled water insoluble capsules that dissolve in acid, that incorporate the aqueous NO solution, and other conventional techniques including, for example, contained in pre-sealed water bottles. A system to that can incorporate NO gas into a liquid for swallowing on demand can also be envisioned.

One skilled in the art can also appreciate additional dosage forms in which to deliver an aqueous solution of NO including but not limited to incorporation of the aqueous NO solution into a stable emulsion or liposomal formulation. One may further envision a formulation whereby the emulsified or lioposmal NO solution is incorporated into a chewable gum where the dose is delivered by chewing the dosage form before, during, or after a meal.

### Small Molecule Therapeutics:

In recent years there has been an increasing interest in therapeutics with multiple modes of action to treat diseases that involve several mediators. These multi-component drugs offer the advantage of an improved therapeutic profile by delivering a multi-pronged attack on the mechanisms that result in a disease state.

Gastrointestinal dysfunction potentially can involve several aberrant conditions such as inappropriate acid secretion, improper musculature control, epithelial erosion or infection. Due to this, an agent that can address more than one of these disease modalities would offer a benefit over a mono-therapy.

Utilization of a nitric oxide donor such as a diazeniumdioloate to normalize muscle tone along with an agent to impact acid secretion and epithelial healing would be beneficial. H₂ receptor antagonists block the action of histamine on parietal cells in the stomach decreasing acid production. Incorporation of a nitric oxide donor into an H₂ receptor antagonist could potentially improve gastric dysfunction. Cimetidine is one such non-limiting exemplary agent in this class that demonstrates a reduction in acid production. As shown in Scheme 1, conjugation of an exemplary H₂ receptor antagonist with a nitric oxide donor can occur through an acid labile linker such as a carbamate after suitably protecting the guanidine by a method that is known to one skilled in the art. In Scheme 1, R₉ can be a substituted or unsubstituted aryl or heteroaryl group. Substituents of R₉ may include but are not limited to electron withdrawing groups (e.g., NO₂, CN, carbonyl, substituted alkyl [e.g. —CF₃]). R₁₀ may be represented by, but is not limited to —CN, an ether group, such as, but not limited to —OCH₃—OCH₂CH₃, and —OSi(CH₃)₃; a tertiary amine; or a thioether, such as but not limited to —SCH₂CH₃ and —SPh (substituted or unsubstituted). Oral administration of this dual-acting agent and exposure to the acidic environment in the stomach would yield the diazeniumdiolate and the H₂ receptor antagonist followed by liberation of nitric oxide from the diazeniumdiolate.

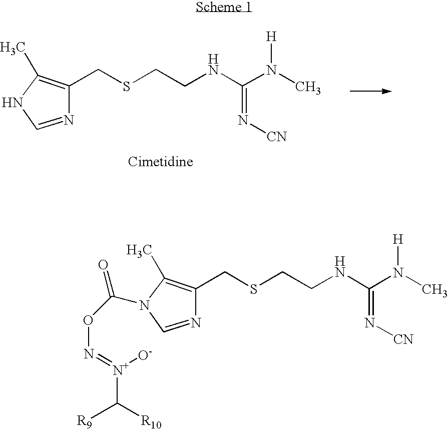

Alternatively, the imidazole of cimetidine can be capped with acetyl chloride to give acetamide after suitably protecting the guanidine by a method that is known to one skilled in the art. This can then be treated with base such as sodium trimethylsilanolate (NaOTMS) followed by treatment with nitric oxide under pressure to give a diazeniumdiolated

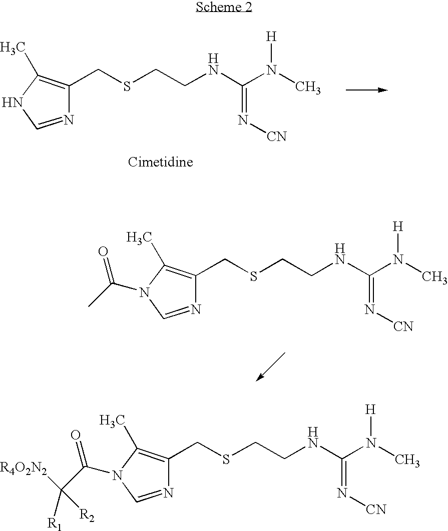

derivative as depicted in Scheme 2 where R₁ and R₂ can be —N₂O₂R₄ or H, and R₄ is an alkali metal ion such as but not limited to Na⁺ and K⁺, or a diazeniumdiolate protecting/capping group or a suitably tethered/attached molecule displaying complementary or synergistic biological activity.

Proton pump inhibitors, which are used to reduce acid secretion by the stomach, can be represented by the general formula depicted in Scheme 3 where R₁₁ can be H, alkyl including but not limited to —CH₃, —CH₂CH₃ and C₃H₇—, fluoroalkyl including but not limited to —CH₂F, —CHF₂, —CF₃, —CH₂CF₃ and —CF₂CF₃ and alkoxy including but not limited to —OCH₃, —OCH₂CH₃, —OC₃H₇, —OCH₂F, —OCHF₂, —OCF₃, —OCH₂CF₃, —OCF₂CF₃ and OCH₂CH₂CH₂OCH₃. Incorporation of the diazeniumdiolate moiety into these drugs will improve the treatment of gastrointestinal dysfunction. As exemplified by the general formula for a proton pump inhibitor and by the proton pump inhibitor omprazole, suitably protecting the guanidine by a method that is known to one skilled in the art and treatment with a base such as NaOTMS followed by exposure to nitric oxide under pressure to give a diazeniumdiolated derivative as depicted in Scheme 3 where R₁ can be —N₂O₂R₄ or H, and R₄ is an alkali metal ion such as but not limited to Na⁺ and K⁺, or a diazeniumdiolate protecting/capping group or a suitably tethered/attached molecule displaying complementary or synergistic biological activity. Other representative proton pump inhibitors are Lansoprazole, Pantoprazole and Rabeprazole.

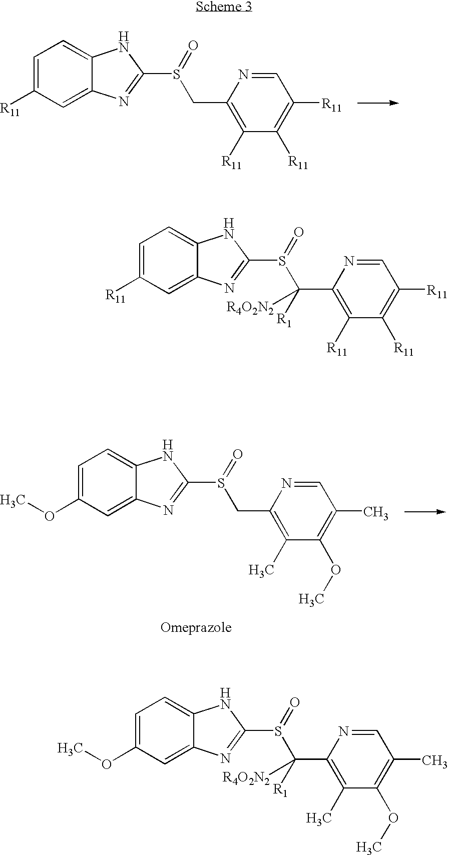

Erythromycin is a macrolide antibiotic that has been shown to be an agonist of the motilin receptor (Smith and Ferris, 2003), and increases the frequency and amplitude of antral contractions of the stomach to remove chyme and residual debris. As such, it has been employed in the treatment of gastric dysfunction resulting in delayed gastric emptying. Augmentation of this activity of erythromycin by the incorporation of diazeniumdiolate moieties into the structure for the release nitric oxide would improve the efficacy of this agent. As shown in Scheme 4, the hydroxyl groups of erythromycin can be protected by

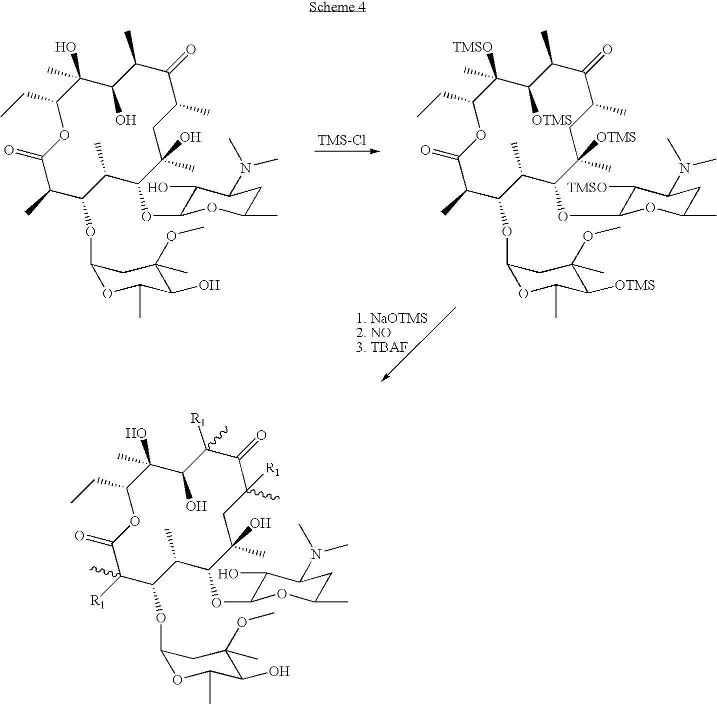

a protecting group such as trimethylsilyl (TMS) by reaction with TMSCl followed by treatment with a base such as NaOTMS to remove the protons alpha to the carbonyls. Exposure to nitric oxide under pressure will then result in the incorporation of diazeniumdiolate moieties into the molecule where R₁, can be —N₂O₂R₄ or H with the proviso that at least one substituent is —N₂O₂R₄, and R₄ is an alkali metal ion such as but not limited to Na⁺ and K⁺, or a diazeniumdiolate protecting/capping group or a suitably tethered/attached molecule displaying complementary or synergistic biological activity. Removal of the TMS protecting groups can then be accomplished through the use of tetrabutylammonium fluoride (TBAF). As shown in Scheme 4, proton abstraction alpha to the carbonyl may result in racemization.

Domperidone is a peripheral D₂ receptor antagonist that is thought to improve antral and duodenal contraction by dopaminergic antagonism of the myenteric plexus. Although not approved in the US, it is used by many countries for the management of gastrointestinal dysfunction such as gastroparesis. Augmentation of this agent with a nitric oxide donor moiety would improve efficacy. As such, domperidone can be acylated under basic conditions to produce a diacetyl derivative as shown in Scheme 5.

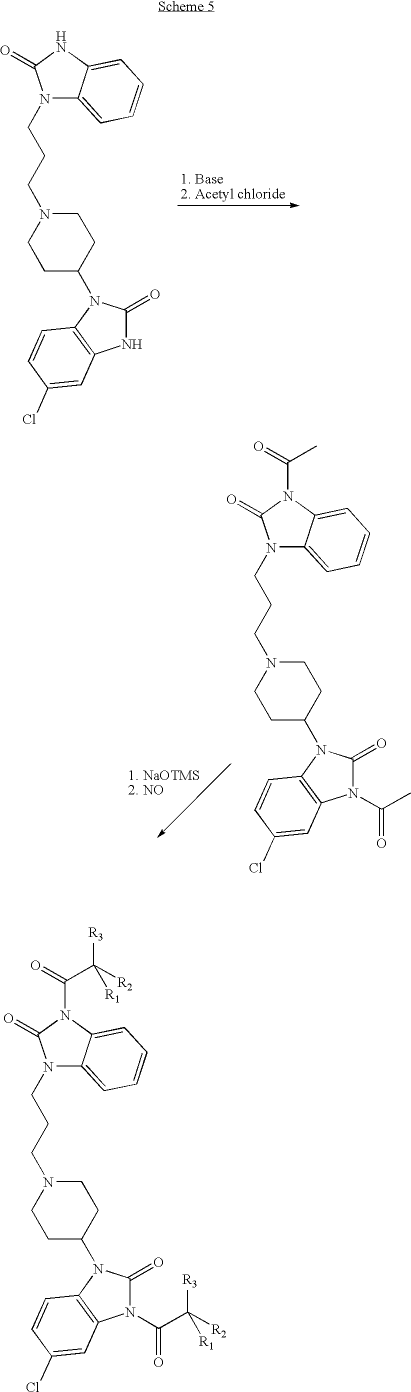

Treatment with a base such as NaOTMS followed by exposure to nitric oxide would yield the diazeniumdiolated derivative where R₁, R₂, R₃ can be —N₂O₂R₄ or H with the proviso that at least one substituent is —N₂O₂R₄, and R₄ is an alkali metal ion such as but not limited to Na⁺ and K^{±}, or a diazeniumdiolate protecting/capping group or a suitably tethered/attached molecule displaying complementary or synergistic biological activity.

In those conditions where inflammation is associated with a gastric motility disorder, a Non-Steroidal Anti-inflammatory Drug (NSAID) that has been modified as shown in Scheme 6, by a diazeniumdiolate group would prove beneficial. In Scheme 6, R₁₁ can be CH₃ or H and the molecule may be chiral, racemic or achiral. R₁₂ may be CH₃, H or N₂O₂R₄. R₄ is an alkali metal ion such as but not limited to Na⁺ and K⁺, or a diazeniumdiolate protecting/capping group or a suitably tethered/attached molecule displaying complementary or synergistic biological activity. R₁₃ is H, an alkali metal ion such as but not limited to Na⁺ and K⁺ or an alkyl group such as but not limited to methyl, ethyl, propyl, isopropyl, butyl, isobuyl, sec-butyl, t-butyl or methylphenyl

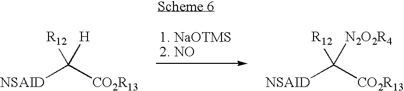

As a non-limiting example, a Naproxen derivative can be subjected to a base such as NaOTMS followed by treatment with nitric oxide to give the diazeniumdiolate analog depicted in Scheme 7a

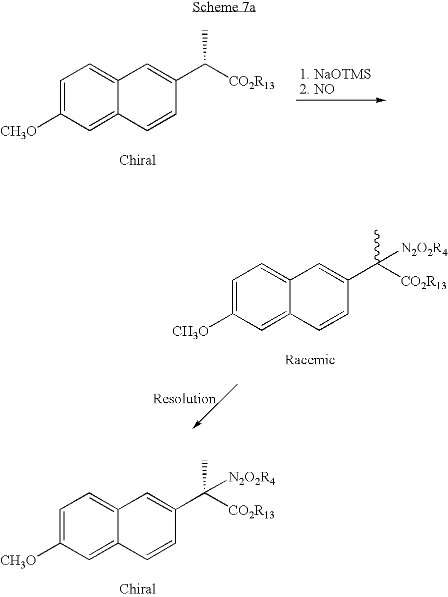

where R₄ is an alkali metal ion such as but not limited to Na⁺ and K⁺, or a diazeniumdiolate protecting/capping group or a suitably tethered/attached molecule displaying complementary or synergistic biological activity R₁₃ is H, an alkali metal ion such as but not limited to Na⁺ and K⁺ or an alkyl group such as but not limited to methyl, ethyl, propyl, isopropyl, butyl, isobuyl, sec-butyl, t-butyl or methylphenyl. Since Naproxen is a chiral molecule (S-isomer), base treatment can result in racemization. In this case, a chiral resolution would have to be used to isolate the more potent R isomer. Techniques such as chiral chromatography or resolution by formation of a diastereomer can be employed in the isolation. This procedure can be applied to other NSAIDS such as arylpropionic acids including but not limited to ibuprofen, ketoprofen and flurbiprofen and arylalkanoic acids such as but not limited to indomethacin, etodolac, ketorolac and sulindac. In cases where the NSAID is not chiral, a resolution step will not be necessary.

Alternatively, an NSAID can be linked via a carboxylic acid ester bond to a suitably tethered diazeniumdiolated moiety as illustrated for Naproxen in Scheme 7b where a suitably activated diazeniumdiolated alcohol, which is illustrated by example with the non-limiting benzylic alcohol, is coupled via a basic reagent to yield an ester. The acidic environment of the stomach will liberate NO from the diazeniumdiolate group while concomitantly cleaving the ester to yield Naproxen. R₁ can be —N₂O₂R₄ or H, and R₄ is an alkali metal ion such as but not limited to Na⁺ and K⁺, or a diazeniumdiolate protecting/capping group or a suitably tethered/attached molecule displaying complementary or synergistic biological activity.

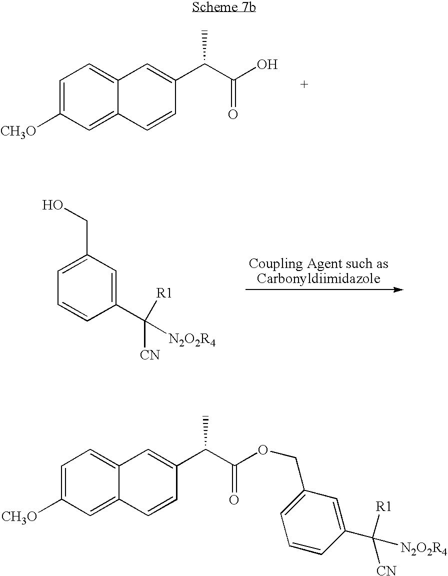

### Use of Nitric Oxide-Releasing Polymers for the Treatment of Gastric Dysfunction: Advantages of Polymers

Polymeric delivery of nitric oxide may have unique advantages over other modes of delivery, including, for example, small molecule delivery. Small molecules are easily absorbed into the systemic circulation. While the body is able to manage sudden increases in NO, NO is a potent vasodilator and rapid release of NO by circulating NO donors do have the potential to cause a precipitous drop in blood pressure. This effect may be additive or synergistic with any medications designed to lower blood pressure that the patient may be taking to treat hypertension. One of the key advantages of polymeric NO delivery include the ability to restrict the NO release to the lumen of the GI cavity without the possibility of systemic absorption of NO donors.

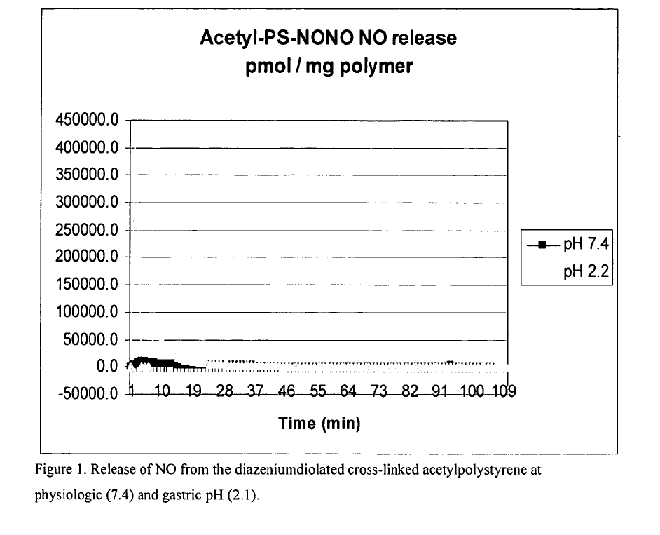

Unlike most small molecule NO donors, polymers can deliver sustained release of NO for pre-determined, controlled and/or extended periods of time. Highly hydrophobic polymers can restrict the access of stomach acid and juices to the active chemical headgroups responsible for the release of NO. This phenomenon is demonstrated by FIG. 1 which shows a comparison of the NO release from the diazeniumdiolated cross-linked acetylpolystyrene at physiologic (7.4) and gastric pH (2.1). This polymer exhibits a large spike in NO release on initial exposure to aqueous solutions. The effect is more pronounced at pH 2.1, as acid is known to accelerate the release of NO from diazeniumdiolates. The spike is likely due to release of NO from the exposed surface of the polymers. After approximately 40 min, the rate of release of NO at both pH levels becomes equivalent, indicating that the release of NO from the diazeniumdiolate groups embedded in the hydrophobic pockets has become diffusion-limited. Thus, hydrophobic polymers can be used to protect NO-releasing donor groups, especially diazeniumdiolate groups, from releasing prematurely at gastric pH levels.

### Advantages of C-Based Diazeniumdiolate Polymers for Use in the Treatment of Neuropathic Gastrointestinal Dysfunction.

One skilled in the art will appreciate that a wide variety of NO-releasing polymers can be used for the treatment of gastric dysfunction. C-based diazeniumdiolate polymers such as those described in PCT/US05/000174 and PCT/US2006/016012, which are incorporated by reference herein in their entireties, have multiple advantages compared to other NO-releasing polymers. The vast majority of polymeric NO donors described are of the nitrogen-based diazeniumdiolates, also known as the N-based diazeniumdiolate class as disclosed in U.S. Pat. No. 5,405,919, Keefer and Hrabie; U.S. Pat. No. 5,525,357, Keefer et al; U.S. Pat. No. 5,632,981, Saavedra et al.; U.S. Pat. No. 5,676,963 Keefer and Hrabie; U.S. Pat. No. 5,691,423, Smith et al.; U.S. Pat. No. 5,718,892 Keefer and Hrabie; U.S. Pat. No. 5,962,520, Smith and Rao; U.S. Pat. No. 6,200,558, Saavedra et al.; U.S. Pat. No. 6,270,779, Fitzhugh et al.; U.S. Patent Application US 2003/0012816 AI, West and Masters; and U.S. Pat. No. 6,382,526, 6,520,425, 6,695,992, and 6,737,447, all of which are also incorporated by reference herein in their entireties. While N-based diazeniumdiolate polymers have the advantages of localized spontaneous and generally controllable release of NO under physiological conditions, a major disadvantage associated with all N-based diazeniumdiolates is their potential to form carcinogenic nitrosamines upon decomposition (Parzuchowski et al., 2002). Many nitrosamines are extremely carcinogenic and the potential for nitrosamine formation limits the N-based diazeniumdiolate class of NO donors from consideration as therapeutic agents based on safety issues. The nitrosamine formation is exacerbated by the low pH of the stomach lumen.

Other non-diazeniumdiolate forms of polymeric NO donors have been described including S-nitroso compounds (U.S. Pat. Nos. 5,770,645 and 6,232,434.) and C-nitroso compounds (U.S. Pat. No. 5,665,077 and U.S. Pat. No. 6,359,182). Regarding the S-nitroso compounds, their therapeutic potential is limited due to their rapid and unpredictable decomposition (release of NO) in the presence of trace levels of Cu(I) and possibly Cu(II) ions (Dicks et al., 1996). Furthermore, S-nitroso compounds may decompose by direct transfer of NO to reduced tissue or food thiols (Liu et al., 1998). Finally, many mammalian enzymes may catalyze the release of NO from S-nitroso compounds (Gordge et al., 1996; Zai et al., 1999). However food levels of ions, enzymes, and thiols are subject to a wide range of variability, making the release of NO unpredictable from meal to meal. The dependence and sensitivity of NO release on blood and tissue components limits the therapeutic potential of nitroso compounds in medicine.

Several references to C-based diazeniumdiolate small molecules (as used herein and throughout this disclosure, small molecules are generally described as molecules with a FormulaWeight of 600 or less) which release NO, have been disclosed (U.S. Pat. No. 6,232,336; 6,511,991; 6,673,338; Arnold et al. 2000; Arnold et al. 2002a; Arnold et al. 2002b). C-based diazeniumdiolates are desirable because in contrast to N-based diazeniumdiolates they are structurally unable to form nitrosamines while maintaining their ability to spontaneously release NO under physiological conditions. Furthermore, there have been recently published reports on NO-releasing imidates, methanetrisdiazeniumdiolates, and a bisdiazeniumdiolates derived from 1,4-benzoquinone dioxime which released 4 moles of NO per mole of compound. (Arnold et al. 2000; Arnold et al. 2002a; Arnold et al. 2002b). While the NO-releasing properties of these small molecules are favorable, small molecules are very difficult to localize in the body after administration and tend to diffuse easily throughout the body, resulting in possible systemic side effects of NO. An additional problem specific to imidate- and thioimidate-derived molecules is that the protein binding properties of imidates may be undesirable in applications involving contact with blood, plasma, cells, or tissue because the imidate may react to form a covalent bond with tissue protein. Protein binding may lead to the inactivation of the protein, an unfavorable distribution of the NO releasing moiety, the creation of antigenic proteins, etc.

### Detailed Description of Nitric Oxide Releasing Polymers for the Treatment of Gastrointestinal Disorders

In one exemplary embodiment, the present invention comprises NO-releasing polymers of the general structure shown in Formula 1. The polymer can be made of any standard polymer backbone. In one embodiment, the polymer is a hydrophobic polymeric backbone (e.g., polystyrene, PET, polymethylmethacrylate). The optional substituent X is a di-, tri- or tetravalent linker group including but not limited to —C(O)—, —OC(O)—, —NHC(O)—, —O—, —S—, —NR₈— (where the R₈ is not H), CR₆(R₇) (where R₆ and R₇ may be an H), or substituted or unsubstituted aliphatic or aryl groups. The optional substituent R is an aliphatic or aryl group, unsubstituted or substituted. Substituents on R may include but are not limited to electron withdrawing groups (e.g., NO₂, CN, carbonyl, substituted alkyl [e.g. —CF₃]). The optional substituent Y is an optional di-, tri- or tetravalent linker group including but not limited to —C(O)—, —OC(O)—, —NHC(O)—, —O—, —S—, —NR₈— (where the R₈ is not H), CR₆(R₇) (where R₆ and R₇ may be an H), or substituted or unsubstituted aliphatic, aryl or heteroaryl group. The R₄ substituent includes but is not limited to an alkali metal ion such as but not limited to Na⁺ and K⁺, or a diazeniumdiolate protecting/capping group or suitably tethered/attached molecule displaying complementary or synergistic biological activity. The polymer would be prepared utilizing a monomer with —R—C(R₁)(R₂)R₃ group, or it may be added after polymerization via coupling to X. The —R—C(R₁)(R₂)R₃ appended polymer could be converted to the C-based diazeniumdiolate using, for example, base in the presence of NO gas.

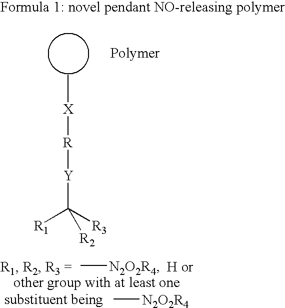

A further embodiment would optionally include the acidic proton containing C group as part of the polymer backbone as shown in Formula 2. The polymer can be made of any standard polymer backbone containing suitable accessible C atoms with acidic protons. In one embodiment, the polymer is a hydrophobic polymeric substrate (e.g., polystyrene, PET, polymethylmethacrylate). Y is a di-, tri- or tetravalent linker group including but not limited to —C(O)—, —OC(O)—, —NHC(O)—, —O—, —S—, —NR₈— (where the R₈ is not an H), CR₆(R₇) (where R₆ and R₇ may be an H), or substituted or unsubstituted aliphatic, aryl or heteroaryl group. The R₄ substituent includes but is not limited to an alkali metal ion such as but not limited to Na⁺ and K⁺, or a diazeniumdiolate protecting group as described in U.S. Pat. No. 6,610,660, or other diazeniumdiolate protecting/capping group or suitably tethered/attached molecule displaying complementary or synergistic biological activity. The substituent R₂ is —N₂O₂R₄, H or other group. The polymer of Formula 2 is converted to the C-based diazeniumdiolate using, for example, base in the presence of NO gas.

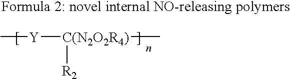

A further embodiment would be to have the acidic proton-containing C groups as multiple sites of activity in each monomer unit as shown in Formula 3. The polymer can be made of any standard polymer backbone containing suitable accessible C atoms with acidic protons. In this embodiment, the polymer is a hydrophobic polymer substrate (e.g., polystyrene, PET, polymethylmethacrylate). The substituent X is a di-, tri- or tetravalent linker group including but not limited to —C(O)—, —OC(O)—, —NHC(O)—, —O—, —S—, —NR₈— (where the R₈ is not an H), CR₆(R₇) (where R₆ and R₇ may be an H), or substituted or unsubstituted aliphatic, aryl or heteroaryl group. Preferably substituent X is an unsubstituted or substituted aliphatic or aryl group. Y may or may not be the same and are a di-, tri- or tetravalent linker group including but not limited to —C(O)—, —OC(O)—, —NHC(O)—, —O—, —S—, —NR₈— (where the R₈ is not an H), CR₆(R₇) (where R₆ and R₇ may be an H), or substituted or unsubstituted aliphatic or aryl groups. The R₄ substituent includes but is not limited to an alkali metal ion such as but not limited to Na⁺ and K⁺, or a diazeniumdiolate protecting group as described in U.S. Pat. No. 6,610,660, or other diazeniumdiolate protecting/capping group or suitably tethered/attached molecule displaying complementary or synergistic biological activity. The substituents R₃, R₅═—N₂O₂R₄, H or other group. The polymer of Formula 3 is converted to the C-based diazeniumdiolate using, for example, base in the presence of NO gas.

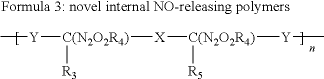

A further embodiment of the invention comprises NO-releasing polymers of the general structure shown in Formula 4. The polymer can be made of any standard polymer backbone. In one embodiment, the polymer is a hydrophobic polymer substrate (e.g., polystyrene, PET, polymethylmethacrylate). X is a di-, tri- or tetravalent linker group including but not limited to —C(O)—, —OC(O)—, —NHC(O)—, —O—, —S—, —NR₈— (where the R₈ is not an H), CR₆(R₇) (where R₆ and R₇ may be an H), or substituted or unsubstituted aliphatic, aryl or heteroaryl group. The pendant aryl group may have one or more substituents G, where G may be H or other groups. The R₁ group may be an —N₂O₂R₄ , H, or other group. The R₄ substituent includes but is not limited to an alkali metal ion such as but not limited to Na⁺ and K⁺, or a diazeniumdiolate protecting group as described in U.S. Pat. No. 6,610,660, or other diazeniumdiolate protecting/capping group or suitably tethered/attached molecule displaying complementary or synergistic biological activity. The polymer could be prepared utilizing a monomer with an attached benzyl group, or it may be added after polymerization. The benzyl appended polymer is converted to the C-based diazeniumdiolate using base in the presence of NO gas.

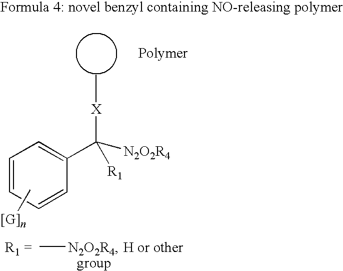

Any of a wide variety of polymers can be used in the context of the present invention. It is only necessary that the polymer selected is biologically acceptable. Illustrative of the polymers suitable for use in the present invention and used as the “Polymer”, “Polymer 1”, or “Polymer 2” (collectively “Polymer”) in the general formulas include, but are not limited to: polystyrene; divinylbenzene cross-linked polystyrene; poly(α-methylstyrene); poly(4-methylstyrene); polyvinyltoluene; polyvinylstearate; polyvinylpyrrolidone; poly(4-vinylpyridine); poly(4-vinylphenol); poly(1-vinylnaphthalene); poly(2-vinylnaphthalene); poly(vinylmethylketone); poly(vinylidene fluoride); poly(vinylbenzyl chloride); polyvinylalcohol; poly(vinylacetate); poly(4-vinylbiphenyl); poly(9-vinylcarbazole); poly(2-vinylpyridine); poly(4-vinylpyridine); polybutadiene; polybutene; poly(butylacrylate); poly(1,4-butyleneadipate); poly(1,4-butyleneterephthalate); poly(ethyleneterephthalate); poly(ethylenesuccinate); poly(butylmethacrylate); poly(ethylene oxide); polychloroprene; polyethylene; polytetrafluoroethylene; polyvinyl chloride; polypropylene; polydimethylsiloxane; polyacrylonitrile; polyaniline; polysulfone; polyethylene glycol; polypropylene glycol; polyacrylic acid; polyallylamine; poly(benzylmethacrylate); derivatized polyolefins such as polyethylenimine; poly(ethyl methacrylate); polyisobutylene; poly(isobutyl methacrylate); polyisoprene; poly(DL-lactide); poly(methylmethacrylate); polypyrrole; poly(carbonate urethane); poly[di(ethylene glycol)adipate]; polyepichlorohydrin; phenolic resins (novolacs and resoles); poly(ethyl acrylate); and combinations thereof including grafts and copolymerizations.

Polymer may also be represented by a styrenic resin, including, but not limited to: acrylonitrile butadiene styrene terpolymer; acrylonitrile-chlorinated polyethylene-styrene terpolymer; acrylic styrene acrylonitrile terpolymer; styrene acrylonitrile copolymers; olefin modified styrene acrylonitrile copolymers; chloromethylpolystyrene polystyrene cross linked with divinylbenzene, styrene butadiene copolymers, and cyanomethylpolystyrene polystyrene copolymer cross linked with divinylbenzene.

Furthermore, Polymer may be represented by a polyamide, including, but not limited to: polyacrylamide; poly[4,4′-methylenebis(phenylisocyanate)-alt-1,4-butanediol/di(propylene glycol)/polycaprolactone]; poly[4,4′-methylenebis(phenyl isocyanate)-alt-1,4-butanediol/poly(butylene adipate)]; poly[4,4′-methylenebis(phenyl isocyanate)-alt-1,4-butanediol/poly(ethylene glycol-co-propylene glycol)/polycaprolactone]; poly[4,4′-methylenebis(phenylisocyanate)-alt-1,4-butanediol/polytetrahydrofuran]; terephthalic acid and isophthalic acid derivatives of aromatic polyamides (e.g. Nylon 6T and Nylon 6I, respectively); poly(imino-1,4-phenyleneiminocarbonyl-1,4-phenylenecarbonyl); poly(m-phenylene isophthalamide); poly(p-benzamide); poly(trimethylhexamethylene terephthalatamide); poly-m-xylyene adipamide; poly(meta-phenylene isophthalamide) (e.g. Nomex); copolymers and combinations thereof; and the like.

Also, Polymer may be represented by polymers including, but not limited to: polyesters; polyarylates; polycarbonates; polyetherimides; polyimides (e.g. Kapton); and polyketones (polyether ketone, polyether ether ketone, polyether ether ketone ketone, and the like); copolymers and combinations thereof; and the like.

Polymer may be represented by a biodegradable polymer including, but not limited to: polylactic acid; polyglycolic acid; poly(ε-caprolactone); copolymers; biopolymers, such as peptides, proteins, oligonucleotides, antibodies and nucleic acids, starburst dendrimers; and combinations thereof.

Diazeniumdiolatation of Benzylic Carbons

A further embodiment of the present invention comprises NO-releasing polymers containing a phenyl group as part of the structure as shown in Formula 5. This embodiment is represented by the general formula:

R—C(R₁)ₓ(N₂O₂R₄)_{y} Formula 5

where y may be 1-3 and x may be 0-2 and the sum of x plus y equals 3, R₁ is not an imidate, thioimidate or amidine. If x is 2, R₁ may be the same or different. R₁ may be represented by, but not limited to an electron withdrawing group such as, but not limited to, a cyano group; an ether group, such as, but not limited to —OCH₃, —OC₂H₅, and —OSi(CH₃)₃; or a thioether, such as, but not limited to, —SC₂H₅, and —SPh (substituted or unsubstituted). The R₁ group may also be a tertiary amine, such as, but not limited to, —N(C₂H₅)₂. R₄ includes but is not limited to Na⁺, K⁺, or a diazeniumdiolate protecting group as described in U.S. Pat. No. 6,610,660, or other protecting/capping group or suitably tethered/attached molecule displaying complementary or synergistic biological activity and R is a phenyl group. The phenyl group may be pendant from the polymer backbone (as shown in Formula 6) or part of the polymer backbone (as shown in Formula 7) or attached to the polymer backbone through linkers as shown previously in Formula 4. In addition to the aforementioned advantages of this technology over the conventional art, manipulation of the R₁ group in Formulas 4 through 7 can alter the release kinetics and the amount of NO released. Alterations of the R₁ group to alter the quantity and kinetics of NO-released are described below.

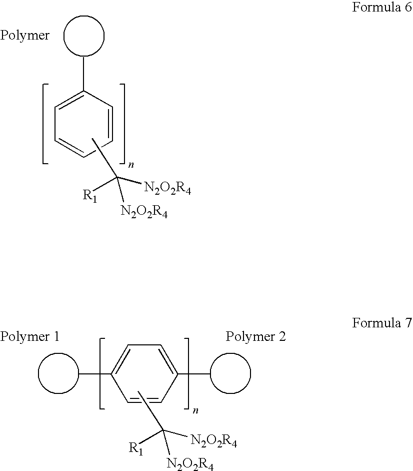

The present invention provides for a novel class of polymeric materials that contain the —[N(O)NO]⁻ functional group bound to a carbon atom. The C-based polymeric diazeniumdiolates of the present invention are useful for a number of reasons. For example, C-based polymeric diazeniumdiolates are advantageous as pharmacological agents, research tools, or as part of a medical device due to their ability to release pharmacologically relevant levels of nitric oxide under physiological conditions without the possibility of forming potent nitrosamine carcinogens. Many of the C-based polymeric diazeniumdiolates of the present invention are insoluble. This property gives this class of materials a number of uses and advantages, including but not limited to delivery of the nitric oxide donating polymer to the gastrointestinal tract by oral ingestion with minimal systemic exposure to NO.

In Formulas 4, 5, 6 and 7, R₁ may not be represented by an imidate, thioimidate or amidine. R₁ may be represented by, but is not limited to an electron withdrawing group such as but not limited to a cyano group; an ether group, such as, but not limited to —OCH₃, —OC₂H₅, and —OSi(CH₃)₃; or a thioether, such as, but not limited to, —SC₂H₅, and —SPh (where the Ph is substituted or unsubstituted). The R₁ group may also be an tertiary amine, such as, but not limited to, —N(C₂H₅)₂, and in another embodiment is a tertiary amine other than an enamine.

The R₄ group in Formulas 1-7 may be a countercation or a covalently bound protecting/capping group or suitably tethered/attached molecule displaying complementary or synergistic biological activity, respectively. In embodiments where the R₄ group is a countercation, the group may be any countercation, pharmaceutically acceptable or not, including but not limited to alkali metals such as sodium, potassium, lithium; Group IIa metals such as calcium and magnesium; transition metals such as iron, copper, and zinc, as well as the other Group Ib elements such as silver and gold. Other pharmaceutically acceptable countercations that may be used include but are not limited to ammonium, other quaternary amines such as but not limited to choline, benzalkonium ion derivatives. As understood by those skilled in the art, the negatively charged diazeniumdiolate group must be counter balanced with equivalent positive charge. Thus, referring to Formula 5, the valence number of the countercation or countercations (R₄) must match the stoichiometric number of diazeniumdiolate groups, both represented by y. In embodiments where more than one diazeniumdiolate is bound to the benzylic carbon, and R₄ is monovalent, R₄ can be the same cation or different cations.

R₄ (Formula 1 through 7) can also be any inorganic or organic group covalently bound to the O²-oxygen of the diazeniumdiolate functional group including but not limited to substituted or unsubstituted aryl groups, as well as a sulfonyl, glycosyl, acyl, alkyl or olefinic group. The alkyl and olefinic group can be a straight chain, branched chain or substituted chain. R₄ (Formula 1 through 7) may be a saturated alkyl, such as, methyl or ethyl or an unsaturated alkyl (such as ally! or vinyl). Vinyl protected diazeniumdiolates are known to be metabolically activated by cytochrome P-450 (Qu et al., 2007). R₄ (Formula 1 through 7) may be a functionalized alkyl, such as, but not limited to, 2-bromoethyl, 2-hydroxypropyl, 2-hydroxyethyl or S-acetyl-2-mercaptoethyl. The latter example is an esterase sensitive protecting group. The former examples provide a chemical functional group handle. Such strategies have been successfully employed to link peptides to the diazeniumdiolate molecule. Hydrolysis may be prolonged by addition of the methoxymethyl protecting group. R₄ (Formula 1 through 7) may be an aryl group, such as 2,4-dinitrophenyl. This type of protecting group is sensitive towards nucleophiles, such as glutathione and other thiols. It is apparent to those skilled in the art that several different protecting groups may be used, and/or the stoichiometry of the protecting group addition may be adjusted such that not all the diazeniumdiolate moieties are protected with the same protecting group, or not all the diazeniumdiolate groups are protected at all. By using different protecting groups, or varying the stoichiometry of the protecting group(s) to diazeniumdiolate ratio, the practitioner may engineer the release of NO to a desired rate. R₄ (Formula 1 through 7) may be a directly attached or linked molecule that exhibits complimentary therapeutic activity by acting at another disease modifying biological pathway.

R (Formula 5) is a phenyl group. The phenyl group may be pendant from the polymer backbone (as shown in Formula 6) or part of the polymer backbone (as shown in Formula 7). In non-polymeric embodiments R may be a substituted or non-substituted phenyl group.

Embodiments with Pendant Phenyl Groups

The pendant phenyl ring from the polymer may have substitutions. The substituted phenyl may be substituted with any moiety that does not interfere with the NO-releasing properties of the polymer and maintains a covalent bond to the polymer backbone. Appropriate moieties include, but are not limited to, aliphatic, aromatic and non-aromatic cyclic groups. Aliphatic moieties are comprised of carbon and hydrogen but may also contain a halogen, nitrogen, oxygen, sulfur, or phosphorus. Aromatic cyclic groups are comprised of at least one aromatic ring. Non-aromatic cyclic groups are comprised of a ring structure with no aromatic rings. The phenyl ring may also be incorporated in multi-ring systems examples of which include, but are not limited to, acridine, anthracene, benzazapine, benzodioxepine, benzothiadiazapine, carbazole, cinnoline, fluorescein, isoquinoline, naphthalene, phenanthrene, phenanthradine, phenazine, phthalazine, quinoline, quinoxaline, and other like polycyclic aromatic hydrocarbons. Additional moieties that can be substituted on the phenyl ring include, but are not limited to, ammonium, alkoxy, acetoxy, aryloxy, acetamide, aldehyde, benzyl, cyano, nitro, thio, sulfonic, vinyl, carboxyl, nitroso, trihalosilane, trialkylsilane, trialkylsiloxane, trialkoxysilane, diazeniumdiolate, hydroxyl, halogen, trihalomethyl, ketone, benzyl, and alkylthio.

Polymers according to the present invention may be derived from commercially available linear or divinylbenzene cross-linked chloromethylated polystyrene. Alternatively, chloromethylated polystyrene may be synthesized in a number of ways, including, but not limited to: utilizing chloromethyl alkyl ethers in the presence of Lewis acid catalysts (Merrifield, 1963); oxidation of poly(4-methylstyrene) using cobalt(III) acetate in the presence of lithium chloride (Sheng and Stover, 1997); or treatment of p-methylstyrene with sodium hypochlorite solution in the presence of phase transfer catalysts (Le Cane et al., 2000).

In one exemplary embodiment of the present invention, using Formula 6, a polymer may be synthesized in a two-step procedure as outlined in Scheme 8. In the first step (1), chloromethylated polystyrene (divinylbenzene cross-linked) is modified with known methods to replace the —Cl atom with a nucleophilic substituent. It is desirable that the nucleophilic substituent activates the benzylic carbon protons for the introduction of diazeniumdiolate functional groups. In another embodiment of this invention, the atom replacing the —Cl atom of the chloromethylated polystyrene is an electronegative heteroatom. It is preferred that the nucleophilic group replacing the —Cl atom is electron withdrawing. It is most preferred that the substituent be a cyano group. Additional substituents may be selected from a group that includes —OR, and —SR. The —OR group may be, but is not limited to, —OCH₃, —OC₂H₅, and —OSi(CH₃)₃. The replacing group may be a thiol group, such as, but not limited to, —SC₂H₅, and —SPh (where the Ph group is substituted or unsubstituted).

The second step (2) in Scheme 8 requires treatment of the polymer with a base in the presence of NO gas. The solvent for the reaction should not react with NO in the presence of a base (e.g. acetonitrile or ethanol). It is preferable that the selected solvent should swell the polystyrene. Suitable solvents include, but are not limited to, THF and DMF. Suitable bases include, but are not limited to, sodium methoxide, sodium trimethylsilanolate, and potassium tert-butoxide. In accordance with the method of the invention, the resulting resin derived from chloromethylated polystyrene following these procedures will contain multiple —[N(O)NO]⁻ functional groups which spontaneously release NO in aqueous media. The R₄ substituent referred to in Formulas 5, 6, 7 and Scheme 9 represents a pharmaceutically acceptable counterion, hydrolysable group, biologically active capping group or enzymatically-activated hydrolysable group as described above.

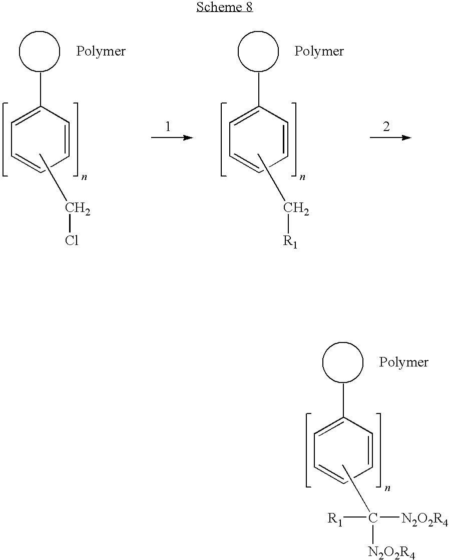

Embodiments with Polymeric Backbone Comprising Phenyl Groups

The polymeric NO releasing resin described in various examples above has the —[N(O)NO]⁻ functional groups pendant to the polymeric backbone. The present invention also provides methods to modify any phenyl ring found in the backbone of the polymer. Thus, other techniques to introduce the nucleophile to obtain the molecular arrangement shown in Formula 5 are considered within the scope of the present invention.

Considering Formula 7, Polymer 1 and Polymer 2 may be equivalent or different from each other, and may include but not be limited to: polybutylene terephthalate; polytrimethylene terephthalate; and polycyclohexylenedimethylene terephthalate. In addition, aramides (a class of polymers in the nylon family synthesized from the reaction of terephthalic acid and a diamine) may also be represented by Polymer 1 or Polymer 2. Examples of such aramides include, but are not limited to, poly(p-phenylene terephthalamide) and poly(m-phenylene isophthalamide). As in other embodiments of this invention described above, it is desirable that the nucleophilic substituent activates the benzylic carbon protons for the introduction of diazeniumdiolate functional groups.

In an exemplary embodiment, the atom replacing the —Cl atom of the chloromethylated polystyrene is an electronegative heteroatom. It is preferred that the nucleophilic group replacing the —Cl atom is electron withdrawing. Preferred substituents for R₁ may be represented by, but are not limited to: a cyano group; an ether group, such as, but not limited to —OCH₃, —OC₂H₅, and —OSi(CH₃)₃; a tertiary amine; and a thioether, such as, but not limited to, —SC₂H₅, and —SPh (where the Ph group can be substituted or unsubstituted). The R₁ group may also be a tertiary amine such as, but not limited to, —N(C₂H₅)₂.

Polyethylene terephthalate (PET) is used in an exemplary embodiment of the present invention, where Polymer 1 and Polymer 2 in Formula 7 represent the repeating ethylene-terephthalate structure. Condensation of terephthalic acid and a diol such as ethylene glycol results in the polyester. Other examples of polyesters can be produced by variation of the diol. Such polyesters may be transformed into NO-releasing materials in a four step process.

By way of example and not in limitation, as shown in Scheme 9, the aromatic ring contained in a polymer of PET may be treated with formaldehyde and acetic acid to produce a benzyl alcohol (Yang, 2000). Treatment with tosyl chloride introduces an effective leaving group onto the polymer. Further treatment with a nucleophile of choice will displace the tosylate and provide the necessary structure for introduction of the —[N(O)NO]⁻ functional group. Therefore, it should be apparent to one of ordinary skill in the art that there may be a wide variety of polymers containing an aromatic phenyl group which may be modified to contain the necessary chemical structure for transformation into a carbon-based diazeniumdiolate through the teachings of the present invention.

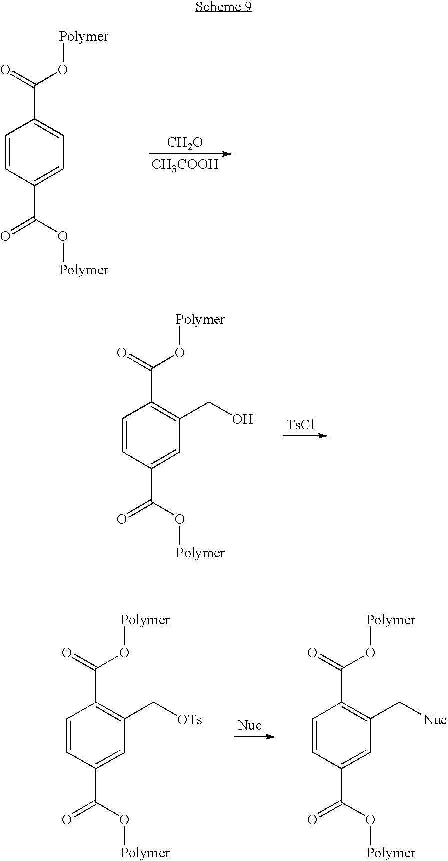

General Chemistry and Strategies to Control Release of NO from Benzylic Embodiments of Formulas 1, 5, 6 and 7

Without restraint to any one theory, the importance of the benzylic structure (methylphenyl group) to the invention is at least threefold. First, the benzylic carbon has relatively acidic protons and the choice of nucleophile should increase the acidity of the benzylic protons such that a base can easily extract a proton. Exposure of benzylic compounds to NO gas in the absence of base is not known to add the diazeniumdiolate functional group. Secondly, the aromatic ring resonance stabilizes the carbanion formed by extraction of a proton by base. The stabilized carbanion allows for the reaction of the carbanion with NO, to produce a radical center and nitroxyl anion (NO⁻). Further reaction of the radical center with NO produces the diazeniumdiolate functional group. The anionic diazeniumdiolate functional group enhances the acidity of the last benzylic proton and allows an additional diazeniumdiolate group to be added to the carbon. In this manner, up to three diazeniumdiolate functional groups are introduced into the polymer via the benzylic carbon. Thirdly, the presence of resonant electrons in the aromatic ring helps promote spontaneous decomposition of the —[N(O)NO]⁻ group in aqueous media. Other bis-diazeniumdiolates, namely methylene-bis-diazeniumdiolate [H₂C(N₂O₂Na)₂] lack resonant electronic forces that participate in the decomposition process and thus show remarkable stability (inability to release NO) in solution (Traube, 1898).

In addition to their advantage of releasing NO under physiological conditions without forming nitrosamine carcinogens, the degree and rate of NO release of these polymeric materials may be engineered using several types of manipulations. Using the basic structure in Formula 6, comparison of M-NONO (where the R₁ in Formula 6 is CN) and a congener where the R₁ is an ethoxy group results in NO release profiles where the cyano-modified polymer exhibits a rapid release profile, whereas the ethoxy-modified polymer exhibits a prolonged but less robust release of NO. Several more examples of the results of manipulation of R₁ of Formula 6 on NO release properties can be examined by comparing the release data in Examples 4 through 6 below. It should be apparent to one skilled in the art that a contiguous polymer may contain more than one type of nucleophilic substituent. As shown in Scheme 10, chloromethylated polystyrene cross-linked with divinylbenzene can be modified with two different nucleophiles, R₁ₐ and R_{1b}, to produce two different types of NO-donor moieties. The ability to control the release rate of NO through manipulation of R₁ allows for precise engineering of the release of NO from the polymer on a macro scale.

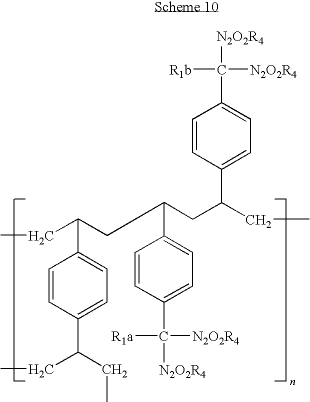

Another exemplary way of reaching the desired amount and rate of NO release on a macro scale is to blend two or more of the individually synthesized polymers together to achieve the desired rate of NO release from the polymer. This method has the advantage over manipulating R₁ in the NO-releasing headgroups of a single polymer because it eliminates the need for stoichiometric control of the synthetic chemistry to achieve the desired release rate. However, this method may not be easily amenable to micro- and nano-scale applications.

An additional way to affect the rate and degree of NO release from the polymer, one which especially holds relevant for the polystyrene-based polymers, is to vary the degree of cross-linking of the polymer. Generally, a lesser degree of cross-linking provides a more porous polymeric structure. While this does not change the degree of nucleophilic substitution and diazeniumdiolation, it provides a more rapid and greater degree of NO release from the polymer because the active NO-releasing sites are more accessible to the aqueous solvent. Increasing the polymer cross-linking decreases the porosity of the polymer, which serves to inhibit aqueous solvent access. Highly cross-linked polymers release NO for longer periods of time (U.S. Pat. No. 6,703,046). Thus, various rates of NO-release may be obtained by controlling the access of aqueous solution to the —[N(O)NO]⁻ functional groups through the degree of cross-linking of the polymer.

The C-based diazeniumdiolate polymer of the present invention is also an improvement over conventional products in terms of time of synthesis and amount of NO generated. For example, according to the teachings of U.S. Pat. No. 5,405,919, a polyamine was linked to chloromethylated polystyrene and a slurry of the aminopolystyrene in acetonitrile was subsequently exposed to NO to produce a N-based diazeniumdiolate. However, such an N-based diazeniumdiolate required a week to synthesize and produced very low levels of NO under physiological conditions which is not useful for many applications. The method of the present invention utilizes a suitable solvent to swell the resin and adding potassium iodide as a catalyst to accelerate the nucleophilic substitution reaction, which is a significant improvement over the reaction time (2 days versus 8 days) and NO-release levels (ppm NO versus very low levels) when compared to that disclosed in U.S. Pat. No. 5,405,919.

### Use of Nitric Oxide-Releasing Polymers for the Treatment of Gastroparesis—Methods for Pharmaceutical Delivery of the Polymer.

Many of the aforementioned polymers can release NO for up to 60 days. This is well beyond the duration of NO release required for treatment of gastroparesis, as normal intestinal transit time is about 24 h. Thus, polymers with extended duration of NO release such as those derived from cross-linked polystyrenes will continue to release NO through the entire length of the gastrointestinal tract. NO release further down the gastrointestinal tract may be beneficial for certain individuals, especially those with nitrergic neuropathies extending throughout the length of the gastrointestinal tract. However, extended duration of NO release throughout the length of the GI tract may result in the manifestation of side effects in other patients. One skilled in the art is aware of a wide variety of methods that can be used to precisely deliver a therapeutic agent over a very specific period of time. Non-limiting examples of these methods are described in further detail below.

One non-limiting method of controlling the release of NO used by those skilled in the art is to vary the hydrophobicity of an NO-releasing polymer, such as, for example, that described in U.S. Pat. No. 6,270,779. It is known that an ultra-long duration NO-releasing polymer can be made by increasing the degree of cross-linking, and therefore the hydrophobicity, of the polymer. Conversely, decreasing the degree of cross-linking will decrease the duration of NO release from the polymer. Thus, one skilled in the art can develop a polystyrene-based or other polymer that is cross-linked to a degree where the NO is released for a certain time period, including but not limited to 6 h, thus limiting the exposure of the remaining length of the digestive tract to NO.

Another non-limiting method to control the duration of NO release for polymers is by size. The rate of NO release from C-based diazeniumdiolates and other NO-releasing polymers is highly dependent on the rate of aqueous penetration into the polymer. This rate can be determined experimentally for any polymer, and the size of the polymer can be adjusted so as to release NO for a limited period of time. A non-limiting example would be an NO-releasing polymer with an aqueous penetration rate of 4 microns (μm) per hour formulated to a particle radius of 24 μm. Such a formulation would be expected to deliver NO for 6 h.

Another non-limiting example would be the use of solid polymeric particles that have surface diazeniumdiolate groups but no groups embedded in hydrophobic pockets. These surface diazeniumdiolate groups are highly accessible and tend to release their entire NO loading rapidly. These diazeniumdiolated particles can then be treated with coatings that hydrolyze at highly specific rates when ingested. The diazeniumdiolated polymer particles can be coated with 0 to a plurality of layers (or zero to any thickness) of the hydrolyzable coating to allow the release of NO at a specific time point. An additional embodiment would use a dosage form comprised of a mixture of diazeniumdiolated particles, factions of which are coated with 0, 1, 2, 3, etc. layers or thickness units of a hydrolyzable coating. This mixture of diazeniumdiolated particles coated with varying layers or thicknesses of hydrolyzable coating can be combined in a single dosage form to allow NO to be released over a specified range of time. A non-limiting example of an appropriate range of time for NO release in the treatment of gastroparesis would be 0 to 6 h.

One skilled in the art knows there are extensive materials and methods that can be used to control the release of drugs in the gastrointestinal tract. A non-limiting list of major microencapsulation techniques includes suspension polymerization, emulsion polymerization, dispersion polymerization, precipitation polymerization, suspension polycondensation, dispersion polycondensation, precipitation polycondensation, interfacial polycondensation, suspension cross-linking, coacervation/phase separation, solvent evaporation/extraction, polymer precipitation, polymer chelation, polymer gelation, polymer melt solidification. The materials may include but are not limited to polysaccharides such as cellulose, agarose; proteins such as albumin, fibrinogen, gelatin and hemoglobin; and an astonishingly wide variety of synthetic polymers. One skilled in the art can determine the ideal formulation including the appropriate materials or mixture of materials, degree of cross-linking if any, and method or methods.

### Dosage Forms

NO-releasing small molecules or polymers of the present invention may be incorporated into a wide variety of pharmaceutically acceptable dosage forms, including but not limited to capsules, tablets, powders, solutions, suspensions, emulsions, liquid-filled capsules, gums, suppositories and other delivery vehicles apparent to those skilled in the arts. The density of the solid dosage form can be varied to target different regions of the stomach. Polymers can be made dense enough to target the dosage form to the lower regions of the stomach and pylorus. One skilled in the art can envision dosage forms that use very dense materials that will allow the dosage form to sink through the stomach chyme to the greater curvature of the antrum and/or the pylorus. A non-limiting exemplary embodiment of such a dosage form could include a dense core material comprised of high density inert polymer, metal, ceramic or other dense material surrounded by an NO-releasing polymer. One skilled in the art can envision additional embodiments that can be used to target the dosage form to different parts of the stomach. Conversely, the dosage form can be made less dense than water (or chyme) to target the upper parts of the stomach and gastroesophogeal sphincter. One skilled in the art can envision a styrofoam-like core supporting the NO-releasing polymer.

Many patients suffering from gastroparesis are unwilling to swallow pharmaceuticals comprised of solid dosage forms. A useful exemplary embodiment of a dosage form for use with the current invention would be the incorporation of the NO-releasing small molecules or polymers into a chewable dosage form whereby the dosage form is chewed by the patient and the NO is delivered in swallowed saliva. Many options for the manufacture of chewable pharmaceutical dosage forms are available to one skilled in the art. These may include but are not limited to U.S. Pat. No. 7,101,579, U.S. Pat. No. 6,986,907.

### Examples

### Example 1

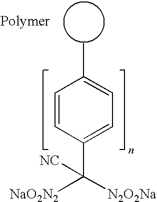

This example provides a method to convert commercially available divinylbenzene cross-linked chloromethylpolystyrene into a nitrile, which is subsequently treated with NO to form acarbon-based diazeniumdiolate. A 50 ml aliquot of DMF is dried over sodium sulfate and then the pre-dried solvent is used to swell 2.37 g (4.42 mmol Cl per g) of chloromethylated polystyrene. After 30 minutes, 3.39 g (52 mmol) KCN and 0.241 g (1.4 mmol) of KI are added. The solution is heated to 60° C. overnight. During this time the resin changes from off white to brick red in color. The resin is washed consecutively with 20 ml portions of DMF, DMF:H₂O, H₂O, EtOH and Et₂O and allowed to air dry. The disappearance of the —CH₂—Cl stretch at 1265 cm-1 and appearance of the nitrile absorption at 2248 cm-1 is indicative of substitution. Diazeniumdiolation: In a Parr pressure vessel, the modified resin-CN is added to 20 ml DMF. This solution is slowly stirred and treated with 20 ml (20 mmol) of 1.0 M sodium trimethylsilanolate in THF. The vessel is degassed and charged with 54 psi NO gas. The head space is flushed with argon and the resin was washed with water, methanol and ether. The tan/slightly orange product was allowed to air dry. This method of diazeniumdiolation avoids the possibility of imidate formation that results when using an alkoxide as the base.

Measurement of NO release: A measured weight of the polymer was assessed for NO release according to the method of Smith et al. (1996) with the exception of performing all current measurements at 37° C. The release rate of NO over time is shown in the table below.

| Time | NO in |
|---|---|
| (min) | pmol/mg/min |
| 0 | 106.9. |
| 5 | 10040.1 |
| 10 | 5320.0 |
| 20 | 3324.9 |
| 30 | 2294.6 |
| 40 | 1680.6 |
| 50 | 1332.0 |
| 60 | 1017.4 |
| 70 | 855.1 |
| 80 | 754.4 |

### Example 2

This example provides a method to convert commercially available divinylbenzene cross-linked chloromethylated polystyrene into a carbon-based diazeniumdiolate including a —OCH₃ group.

To a 50 ml solution of 1:1 DMF/MeOH, the following are added: 1.0 g divinylbenzene cross-linked chloromethylated polystyrene (4.38 mmol Cl/g), 0.014 g KI (0.08 mmol), and 1.0 ml 25% NaOMe (4.37 mmol). The solution is stirred at room temperature overnight. It is then vacuum filtered and washed with MeOH and ether. The product's total weight of 1.0 g is slightly higher than the 0.979 g theoretical weight.

Diazeniumdiolation: The resin-OCH₃ is put in a Parr pressure vessel and 50 ml of 1:1 DMF/MeOH is added. While stirring, 2.0 ml 25% NaOMe (8.76 mmol) is added. The solution is degassed by alternating cycles of inert gas pressurization/venting before exposure to 50 psi NO gas. The consumption of NO gas, an indication of the reaction of the gas with the resin, is determined the next day. In one example, it was observed that 10 psi of NO gas was consumed. After vacuum filtration, washing and air drying, the weight gain is observed. Even in the absence of weight gain, the composition produced can have a positive Greiss reaction as well as NO release, as detected by chemiluminescence.

### Example 3

This example provides a method to convert commercially available divinylbenzene cross-linked chloromethylated polystyrene into a carbon-based diazeniumdiolate including an —OC₂H₅ group. To a 50 ml solution of 1:1 DMF/EtOH, the following are added: 1.0 g divinylbenzene cross-linked chloromethylated polystyrene (4.38 mmol Cl/g), 0.016 g KI (0.09 mmol), and 1.7 ml 24% KOEt (4.38 mmol). The solution is stirred overnight at room temperature. It is then vacuum filtered and washed with EtOH and ether. In one example, the observed weight was 1.22 g, which was slightly more than the expected 1.04 g.

Diazeniumdiolation: The resin-OC₂H₅ is placed in a Parr pressure vessel with 50 ml solution of 1:1 DMF/MeOH, and 2.0 ml of 25% NaOMe (8.76 mmol) is added. The vessel is degassed and exposed to 60 psi NO gas overnight. The resin is then washed with methanol and ether, and air dried. In one example, this material had a positive Greiss reaction and spontaneously generates NO under physiological conditions, as detected by a chemiluminescent NO detector.

### Example 4

This example provides a method to convert commercially available divinylbenzene cross-linked chloromethylated polystyrene into a carbon-based diazeniumdiolate including an —SC₂H₅ group.

In a fume hood, to 50 ml of dried DMF, the following are added: 1.00 g divinylbenzene cross-linked chloromethylated polystyrene (4.42 mmol Cl/g), 40 mg (0.24 mmol) potassium iodide and 372 mg (4.42 mmol) sodium ethanethiolate. This mixture is stirred at room temperature for 72 hours. It is filtered and washed with 25 ml portions of 1:1 DMF:MeOH, MeOH and Et₂O and allowed to air dry.

Diazeniumdiolation: To one gram of resin-SC₂H₅ in a Parr pressure vessel, the following are added: 25 ml of THF and 2.0 ml (8.84 mmoles) of 25% sodium methoxide. The mixture was degassed by alternating charging and discharging the pressure vessel with argon before exposure to 60 psi NO gas overnight. The resin is filtered and washed with 50 ml of 0.01 M NaOH, ethanol and diethyl ether. The resulting resin produces a positive Greiss reaction. When measured in a chemiluminescent NO detector, 100 mg of resin produced 4.1×10⁻¹¹ moles NO/mg resin/min in pH 7.4 buffer at room temperature over a 1 hr period.

### Example 5

This example provides a method to convert commercially available divinylbenzene cross-linked chloromethylated polystyrene into a carbon-based diazeniumdiolate including a —OSi(CH₃)₃ group. In 50 ml of dried DMF, the following are added: 1.00 g divinylbenzene cross-linked chloromethylated polystyrene (4.42 mmol Cl/g), 10 ml of 1.0 M (10 mmoles) sodium trimethylsilanolate and 100 mg (0.6 mmoles) potassium iodide. The mixture is heated to 100° C. for 24 hours. Thereafter, the resin is filtered and washed with 20 ml portions of DMF, MeOH and diethyl ether and allowed to dry in air.

Diazeniumdiolation: the following are placed in a Parr pressure vessel: 1.0 g of modified resin, 30 ml DMF and 2.0 ml (8.84 mmoles) 25% sodium methoxide. The pressure vessel is degassed and then exposed to 60 psi NO for 24 hours. The resin is then filtered and washed consecutively with DMF, MeOH and diethyl ether. Thereafter the resin is dried in air and produces a positive Greiss reaction. When measured in a chemiluminescent NO detector, 100 mg of resin produced 4.1×10⁻¹¹ moles NO/mg resin/min in pH 7.4 buffer at room temperature over a 40 min period.

### Example 6

This example provides a method to convert commercially available divinylbenzene cross-linked chloromethylated polystyrene into a carbon-based diazeniumdiolate including a diethylamine group.

A 2.17 g sample of divinylbenzene cross-linked chloromethylated polystyrene (4.42 mmol Cl⁻/g) is added to 50 ml of DMF. To this suspension, the following are added: 0.123 g (0.74 mmol) KI and 5 ml (72 mmol) diethylamine. The suspension is stirred at 45° C. for 24 hours and then filtered and washed twice with DMF, MeOH and ether. The resin is allowed to air dry.

Diazeniumdiolation: The following are added to a Parr pressure vessel: 100 ml MeOH, 1.0 g modified resin and 2.0 ml (8.7 mmol) 25% NaOMe. After degassing, the solution is exposed to 60 psi NO gas for 24 hours. The resin is then filtered and washed with methanol and ether and allowed to air dry. Over a 150 min period, 100 mg of resin produced 9.3×10⁻¹¹ moles NO/mg resin/min in pH 7.4 buffer at room temperature.

### Example 7

This example demonstrates that the NO derived from the diazeniumdiolated cyanomethylpolystyrene material in Example 1 originates from NO donor groups attached to the resin and not to delocalized free NO gas molecules trapped in the interstitial spaces.

A general concern working with these materials is the possibility of NO becoming trapped in the interstitial spaces within the resin, which can skew the total amount of NO produced from the resin. As a control experiment, 0.50 g of Merrifield resin is placed in 40 ml of a 1:1 DMF/MeOH solution, degassed and exposed to 80 psi NO gas for 24 hours. The resin was then filtered, washed several times with MeOH, acetone and ether. After drying in air, a 50 mg sample was placed in 5 ml of Greiss reagent, which would colorimetrically reveal the presence of any nitrite, a known breakdown product of NO The reagent did not turn the characteristic purple color indicative of the presence of nitrite. Therefore, the NO that is detected from the resin is due to the formation of NO donor groups and not to trapped NO.

### Example 8

This example provides a method to convert a polymer containing an aromatic ring in the backbone of the polymer e.g. poly(ethylene terephthalate) (PET) into a carbon-based diazeniumdiolate.

In a 150 ml beaker, 2.0 g of PET pellets (Sigma-Aldrich, Milwaukee, Wis.) are treated with 10 ml of acetic acid and 10 ml of 37 wt % formaldehyde. The reaction is allowed to stir for 24 hours. The hydroxylated PET is then filtered and washed with three 25 ml portions of water and dried at 100° C. for one hour.

The hydroxylated PET is then suspended in 50 ml of pyridine, chilled in an ice bath, and treated with 4.67 g (2.4×10⁻² mol) of p-toluenesulfonyl chloride. Two minutes after the addition of the p-toluenesulfonyl chloride the reaction is allowed to warm to room temperature. After twenty-four hours, the reaction is filtered and washed with two portions (25 ml) of dried DMF.

The tosylated PET is then placed in 25 ml of dried DMF and 2.03 g (3.1×10⁻² mol) of KCN is added with gentle stirring. After twenty-four hours, the cyanomethylated PET is filtered and washed with DMF (25 ml), 1:1 DMF:H₂O (25 ml), H₂O (2×25 ml), and MeOH (2×25 ml).

The cyanomethylated PET is then placed in a 300 ml Parr pressure vessel to which 25 ml of MeOH is added. The suspension is gently stirred and 1.0 ml of a 1.0 M solution of sodium trimethylsilanolate in tetrahydrofuran is added to the suspension. The pressure vessel is purged and vented 10 times with argon and then charged with NO (80 psi). After twenty-four hours the diazeniumdiolated PET is filtered and washed with 25 ml of EtOH and 25 ml of Et₂O.

### Example 9

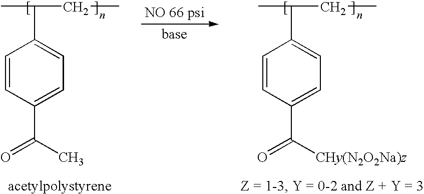

This example converts known acetylpolystyrene to the C-based diazeniumdiolate. To a 300 mL Ace pressure bottle was added 0.25 g acetylpolystyrene resin cross linked with divinylbenzene, followed by 25 mL THF and 0.112 g sodium trimethylsilanolate (NaOTMS), respectively. The vessel was degassed with Ar gas and pressurized with 66 psi NO gas and gently shaken for 18 h. At this time, the vessel was purged with Ar gas and the modified resin was washed with THF, 10 mM NaOH/DMF (1:3), DMF, MeOH, ether and aspirated to dryness to yield a recovery of 0.211 g light yellow beads. In parallel, a control reaction was set up in the same fashion, utilizing 0.100 g resin and 25 mL THF, but no base. The modified resin yields a positive Griess reaction whereas the control sample (no base) yields a negative Griess reaction.

### Example 10

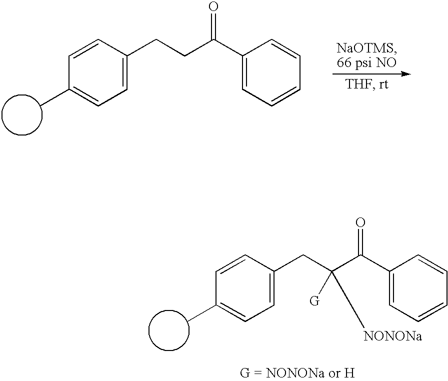

This example converts 3-oxo-3-phenylpropylpolystyrene to the C-based diazeniumdiolate. 3-Oxo-3-phenylpropylpolystyrene was prepared by treatment of Merrifield's resin with acetophenone and NaH in THF at 0 C. The reaction was quenched with MeOH and the resin washed and dried. The presence of the added ketone was confirmed using FT-IR.

Diazeniumdiolation: To a 300 mL Ace pressure bottle was added 0.25 g 3-oxo-3-phenylpropylpolystyrene resin, followed by 25 mL THF and 0.112 g sodium trimethylsilanolate (NaOTMS), respectively. The vessel was degassed with Ar gas and pressurized with 66 psi NO gas and gently shaken for 18 h. At this time, the vessel was purged with Ar gas and the modified resin was washed with THF, 10 mM NaOH/DMF (1:3), DMF, MeOH, ether and aspirated to dryness to yield a recovery of 0.243 g orange/yellow beads. In parallel, set up a control reaction in the same fashion, utilizing 0.100 g resin and 25 mL THF, but no base. The modified resin yields a positive Griess reaction whereas the control sample (no base) yields a negative Griess reaction.

### Example 11

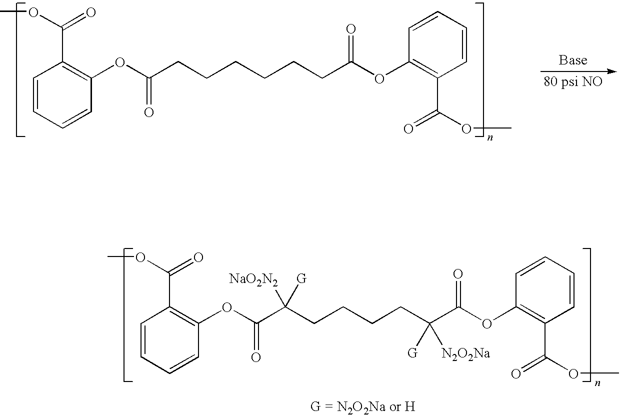

In this example, a so-called “polyaspirin” is utilized as a polymer support for the generation of diazeniumdiolate functionalities in the presence of bulky base and 80 psi NO

Diazeniumdiolation: To a 300 mL Ace pressure bottle was added 6 polymer coated pipette tips, followed by 50 mL DMF and 1.07 g sodium trimethylsilanolate (NaOTMS), respectively. The vessel was degassed with Ar gas and pressurized with 76 psi NO gas and gently shaken for 18 h. At this time, the vessel was purged with Ar gas and the coated pipette tips were washed with THF, ether and aspirated to dryness to yield light yellow coatings. The NO treated pipette tips yielded a positive Griess reaction. NO release was also confirmed utilizing a TEI NOx analyzer in phosphate buffer (0.1 M, pH 7.4).

Polysapirin was developed as a method to deliver aspirin without stomach upset (Schmeltzer et al., 2003) The polymer along with related products are currently being commercialized by Polymerix Corp. (Piscataway, N.J.)

### Example 12

In this Example, the present invention was tested in the form of an oral therapeutic. Adult rats were treated with streptozotocin to destroy their pancreatic beta cells, rendering them diabetic. Seven week diabetic rats, a standard model for diabetic therapeutics, were used to determine the ability of an embodiment of the current invention to reverse the effects of diabetes on liquid gastric emptying time. Rats were given a liquid meal containing a measurable dye, allowing the contents of the stomach to be measured colorimetrically. Non-diabetic rats (Control Group) were fed a dyed meal along with chloromethylated polystyrene modified to substitute a cyano group for the chloride, but not diazeniumdiolated (i.e. does not release NO). Diabetic rats (Diabetic Control) were also fed a dyed meal along with the same non-nitric oxide releasing cyano derivative described in the Control Group. An additional group of diabetic rats were fed a dyed meal along with the nitric oxide-releasing cyanomethylated polystyrene beads described in Example 1 (Diabetic Treated Group). The amount of dyed meal remaining in the stomach after 15 min for each group was determined. The results demonstrating a reversal of the diabetes-induced increase in gastric emptying time by treating with an embodiment of the current invention described in Example 1 above are shown in the Table below.

| | % of Meal Retained |
|---|---|
| GROUP | at 15 min |
| Control | 52.6 ± 4.6 |
| Diabetic Control | 82.6 ± 4.0 |
| Diabetic Treated | 56.6 ± 4.0 |

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention

REFERENCES—The following references, some specifically cited elsewhere in this disclosure, are hereby incorporated by reference herein in their entirety.

Arnold E V, Keefer L K, Hrabie J A. Reaction of nitric oxide with benzyl cyanide to yield a bis-diazeniumdiolated imidate. Tetrahedron Lett 2000, 41:8421-8424.

Arnold E V, Citro M L, Keefer L K, Hrabie J A. A nitric oxide-releasing polydiazeniumdiolate derived from acetonitrile. Org Lett 2002, 4(8):1323-5.

Arnold E V, Citro M L, Saavedra E A, Davies K M, Keefer L K, Hrabie J A. Mechanistic insight into exclusive nitric oxide recovery from a carbon-bound diazeniumdiolate. Nitric Oxide 2002, 7(2):103-8.

Bianco A, Pitocco D, Valenza V, Caputo S, Grieco A, Miele L, Greco A V, and Ghirlanda G. Effect of sildenafil on diabetic gastropathy. Diabetes Care 2002, 25: 1888-1889.

Dicks A P, Williams D L. Generation of nitric oxide from S-nitrosothiols using protein-bound Cu2+ sources. Chem Biol 1996, 3(8):655-9.

Eherer A J, Schwetz I, Hammer H F, Petnehazy T, Scheidl S J, Weber K, Krejs G J. Effect of sildenafil on oesophageal motor function in healthy subjects and patients with oesophageal motor disorders. Gut 2002, 50(6):758-64.

Gordge M P, Hothersall J S, Neild G H, Dutra A A. Role of a copper (I)-dependent enzyme in the anti-platelet action of S-nitrosoglutathione. Br J Pharmacol 1996, 119(3):533-8.

Horowitz M, Dent J, Fraser R, Sun W, and Hebbard G. Role and integration of mechanisms controlling gastric emptying. Dig Dis Sci 1994, 39:7S-13S.

Ishiguchi T, Nakajima M, Sone H, Tada H, Kumagai A K, and Takahashi T. Gastric distensioninduced pyloric relaxation: central nervous system regulation and effects of acute hyperglycaemia in the rat. J Physiol 2001, 533:801-813.

Le Carre E, Lewis N, Ribas C, Wells A. Convenient Preparation of Functionalised Polymer-Based Resins via an Economical Preparation of Chloromethylated Polystyrene Resins (Merrifield Type). Organic Process Research & Development 2000, 4:606-610.

Liu Z, Rudd M A, Freedman J E, Loscalzo J. S-Transnitrosation reactions are involved in the metabolic fate and biological actions of nitric oxide. J Pharmacol Exp Ther 1998, 284(2):526-34.

Mearin F, Camilleri M, and Malagelada J R. Pyloric dysfunction in diabetics with recurrent nausea and vomiting. Gastroenterology 1986, 90:1919-1925.

Mearin F, Mourelle M, Guarner F, Salas A, Riveros-Moreno V, Moncada S, Malagelada J R. Patients with achalasia lack nitric oxide synthase in the gastro-oesophageal junction. Eur J Clin Invest 1993, 23(11):724-8.

Merrifield R B. Solid Phase Peptide Synthesis. I. The Synthesis of a Tetrapeptide. J Am Chem Soc 1963, 85:2149-54.

Micci M A, Kahrig K M, Simmons R S, Sarna S K, Espejo-Navarro M R, and Pasricha P J. Neural stem cell transplantation in the stomach rescues gastric function in neuronal nitric oxide synthase-deficient mice. Gastroenterology 2005, 129:1817-1824.

Orihata M, and Sarna S K. Inhibition of nitric oxide synthase delays gastric emptying of solid meals. J Pharmacol Exp Ther 1994, 271:660-670.

Parkman H P, Hasler W L, and Fisher R S. American Gastroenterological Association medical position statement: diagnosis and treatment of gastroparesis. Gastroenterology 2004, 127:1589-1591.

Parzuchowski P G, Frost M C, Meyerhoff M E. Synthesis and characterization of polymethacrylate-based nitric oxide donors. J Am Chem Soc 2002, 124(41):12182-91.

Qu W, Liu J, Fuquay R, Saavedra J E, Keefer L K, Waalkes M P. The nitric oxide prodrug, V-PYRRO/NO, mitigates arsenic-induced liver cell toxicity and apoptosis Cancer Lett 2007, 256(2):238-45.

Schmeltzer R C, Anastasiou T J, Uhrich K E. Optimized Synthesis of Salicylate-based Poly(anhydride-esters). Polymer Bulletin 2003, 49:441-448.

Shah V, Lyford G, Gores G, Farrugia G. Nitric Oxide in Gastrointestinal Health and Disease. Gateroenterology 2004, 126:903-913.

Sheng Q and Stolver H D H. Selective Functionalization of Poly(4-methylstyrene). Macromolecules 1997, 30:6451-6457.

Sivarao D V, Mashimo H L, Thatte H S, Goyal R K. Lower esophageal sphincter is achalasic in nNOS(−/−) and hypotensive in W/W(v) mutant mice. Gastroenterology 2001, 121(1):34-42.

Smith D F, Ferris C D. Current Concepts in Diabetic Gastroparesis. Drugs 2003, 63(13):1339-1358.

Smith D J, Chakravarthy D, Puffer S, Simmons M L, Hrabie J A, Citro M L, Saavedra J E, Davies K M, Hutsell T C, Mooradian D L, Hanson S R, Keefer L K. Nitric Oxide-Releasing Polymers Containing the [N(O)NO]— Group. J Med Chem 1996, 39:1148-1156.

Traube W. Ueber Synthesen stickstoffhaltiger Verbindungen mit Hülfe des Stickoxyds. Justus Liebig's Annalen der Chemie 1898, 300:81-128.

Watkins C C, Sawa A, Jaffrey S, Blackshaw S, Barrow R K, Snyder S H, and Ferris C D. Insulin restores neuronal nitric oxide synthase expression and function that is lost in diabetic gastropathy. J Clin Invest 2000, 106:373-384.

Yang Z and Chilkoti A. Microstamping of a Biological Ligand onto an Activated Polymer Surface. Adv Mater 2000, 12(6): 413-417.

Zai A, Rudd M A, Scribner A W, Loscalzo J. Cell-surface protein disulfide isomerase catalyzes transnitrosation and regulates intracellular transfer of nitric oxide. J Clin Invest 1999, 103(3):393-9.

1. A method for treating gastrointestinal dysfunction in a mammal by administering a therapeutic amount of a carbon-based diazeniumdiolate compound that delivers nitric oxide and augments nitric oxide signaling.

2. The method of claim 1, wherein the gastrointestinal dysfunction is characterized by hypomotility or hypermotility in at least one of the esophagus, stomach, small intestine, large intestine, colon, or rectum.

3. The method of claim 2, wherein the gastrointestinal dysfunction is further characterized by at least one of nausea, vomiting, heartburn, postprandial discomfort, diarrhea, constipation, indigestion or delayed gastric emptying.

4. The method of claim 1, wherein the gastrointestinal dysfunction is related to at least one of diabetes, anorexia nervosa, bulimia, achlorhydrea, achalasia, anal fissure, intestinal pseudoobstruction, neoplasm, and gastrointestinal damage caused by surgery.

5. The method of claim 4, wherein the gastrointestinal dysfunction is diabetes related.

6. The method of claim 5, wherein the gastrointestinal dysfunction is gastroparesis.

7. The method of claim 4, wherein the intestinal pseudoobstruction is at least one of colonic pseudoobstruction (Ogilivie's syndrome), idiopathic gastroparesis or idiopathic constipation (megacolon).

8. The method of claim 1, wherein the gastrointestinal dysfunction is at least one of hypertrophic pyloric stenosis, functional bowel disease, gastroesophageal reflux disease (GERD), Barrett's metaplasia or Barret's esophagus.

9. A compound for treating gastrointestinal dysfunction in a mammal comprised of a polymeric C-based diazeniumdiolate compound wherein said compound is not an imidate, thioimidate or amidine.

10. The compound of claim 9, wherein said compound releases NO in predictable quantities and wherein said compound does not generate nitrosamines.

11.-32. (canceled)

11. The compound of 10, wherein the compound has a structure as shown in Formula 5 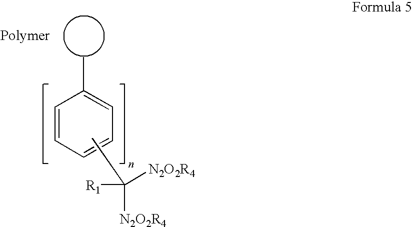
Where R₁ may not be represented by an imidate, thioimidate, or amidine. R₁ may be represented by, but is not limited to an electron withdrawing group such as but not limited to a cyano group; an ether group, such as, but not limited to —OCH₃, —OC₂H₅, and —OSi(CH₃)₃; a tertiary amine; or a thioether, such as, but not limited to, —SC₂H₅, and —SPh (where the Ph is substituted or unsubstituted). R₄ includes but is not limited to an alkali metal ion such as but not limited to Na⁺ and K⁺, a diazeniumdiolate protecting/capping group or suitably tethered/attached molecule displaying complementary or synergistic biological activity,
or the geometric Isomers, enantiomers, diastereomers, and pharmaceutically acceptable salts thereof.

12. The compound of claim 11, wherein the compound has the structure 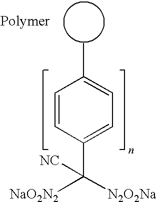
comprises polystyrene that is cross linked with divinylbenzene, or the geometric isomers, enantiomers, diasteromers, and pharmaceutically acceptable salts thereof.

13. The compound of claim 9, wherein the compound is administered orally via a pharmaceutically acceptable dosage form.

14. The compound of claim 13, wherein the dosage form is a controlled release dosage form.

15. The compound of claim 14, wherein the controlled release dosage form involves microencapsulation, membrane permeation, or the like.

16. The compound of claim 15, wherein the oral dosage form is a chewable gum.

39.-48. (canceled)
